(19) United States
(12) Patent Application Publication
(10) Pub. No.: US 2025/0353019 A1
(43) Pub. Date: Nov. 20, 2025

(54) PROTECTION INSERT FOR BIOPROCESS CASSETTES

(71) Applicant: ENTEGRIS, INC., Billerica, MA (US)

(72) Inventors: Jacob Peabody, Roxbury, MA (US); Robert Michael Shydo, Jr., Hampstead, NH (US)

(21) Appl. No.: 19/208,539

(22) Filed: May 14, 2025

Related U.S. Application Data
(60) Provisional application No. 63/648,030, filed on May 15, 2024.

(51) Int. Cl. B01L 9/00; B01L 3/00

(52) U.S. Cl. CPC B01L 9/52; B01L 3/505; B01L 2200/025; B01L 2300/0809; B01L 2300/123

(57) ABSTRACT

A protection insert can be provided with a freezing cassette to secure a bioprocess bag within the cassette. The protection insert can include a crosspiece between first and second arms, and a shielding projection extending from the crosspiece. The first and second arms and the crosspiece can be configured to partially accommodate the bioprocess bag. The crosspiece and/or the shielding projection can include tube channels to direct tubes extending from the bioprocess bag. The crosspiece and/or arms can include a tube resting region, and the tube resting region can include a tube retention feature.

### FIELD

This disclosure is directed to protection inserts for use in cassettes for bioprocess bags.

### BACKGROUND

Cassettes can be used to accommodate bioprocess bags, for example during freezing and thawing operations. When the bioprocess bag is contained within the cassette and a shock event occurs, such as the cassette being dropped or struck, the bag can shift and the bag and/or tubes extending therefrom can be damaged by impacting the interior of the cassette. Breakage of the bag or tubes would cause loss of fluid product from within the bag. The bag and tubes can be particularly susceptible to damage when they are frozen.

### SUMMARY

This disclosure is directed to protection inserts for use in cassettes for bioprocess bags.

By including a protection insert, the position of a bioprocess bag and tubes extending therefrom can be secured within a cassette. The protection insert can reduce the risk of bag or tube breakage, for example during shock events such as the cassette being struck or dropped. This can prevent the loss of highly expensive contents of the bioprocess bag such as drugs, biologics, or the like.

In an embodiment, an apparatus, includes a protection insert. The protection insert includes a crosspiece, a first arm extending from a first end of the crosspiece, and a second arm extending from a second end of the crosspiece. The crosspiece, the first arm, and the second arm are configured to partially accommodate a bioprocess bag. The protection insert further includes a shielding projection extending from the crosspiece, the shielding projection extending on a side of the crosspiece opposite extending directions of the first arm and the second arm and one or more tube channels provided on the crosspiece, each of the one or more tube channels configured to receive at least one of a plurality of tubes extending from the bioprocess bag.

In an embodiment, the apparatus further includes the bioprocess bag and a cassette, the cassette configured to contain the protection insert and the bioprocess bag. In an embodiment, the first arm and the second arm each extend at least half of a length of an internal space of the cassette.

In an embodiment, the first arm and the second arm each include a bag retention segment configured to be positioned between the bioprocess bag and at least one side wall of a cassette when the protection insert is positioned within the cassette and the bioprocess bag is positioned within the protection insert.

In an embodiment, the protection insert includes a plurality of surfaces configured to face the bioprocess bag, and transitions between said plurality of surfaces and adjacent surfaces of the protection insert are rounded.

In an embodiment, at least one of the tube channels is defined at least in part by the shielding projection.

In an embodiment, at least one of the one or more tube channels is configured to direct a respective one of the plurality of tubes extending from the bioprocess bag to follow an arc.

In an embodiment, the protection insert is formed of a polymer.

In an embodiment the protection insert includes an elastomer.

In an embodiment, the crosspiece and at least one of the first arm and the second arm define a tube resting region. In an embodiment, the tube resting region includes a tube retention feature.

In an embodiment, the shielding projection includes a contact surface at a distal end thereof, the contact surface configured to contact an interior wall of a cassette when the protection insert is positioned within said cassette.

In an embodiment, an apparatus includes a cassette configured to accommodate a bag and a protection insert. The protection insert includes a crosspiece, a first arm extending from a first end of the crosspiece and a second arm extending from a second end of the crosspiece. The crosspiece, the first arm, and the second arm are configured to partially accommodate the bag. The protection insert further includes a shielding projection extending from the crosspiece, the shielding projection extending on a side of the crosspiece opposite extending directions of the first arm and the second arm and one or more tube channels provided on the crosspiece, each of the one or more tube channels configured to receive at least one of a plurality of tubes extending from the bag.

In an embodiment, the cassette comprises a first cassette segment and a second cassette segment, wherein the first cassette segment and the second cassette segment are joined by a hinge.

In an embodiment, the first arm and the second arm of the protection insert each extend at least half of a length of an internal space of the cassette.

In an embodiment, at least one of the one or more tube channels is configured to direct a respective one of the plurality of tubes extending from the bioprocess bag to follow an arc.

In an embodiment, the protection insert includes a plurality of surfaces configured to face the bag, and transitions between said plurality of surfaces and adjacent surfaces of the protection insert are rounded.

In an embodiment, the shielding projection includes a contact surface at a distal end thereof, the contact surface configured to contact an interior wall of a cassette when the protection insert is positioned within said cassette.

In an embodiment, the crosspiece and at least one of the first arm and the second arm define a tube resting region. In an embodiment, the tube resting region includes a tube retention feature.

### BRIEF DESCRIPTION OF THE DRAWINGS

FIG. 1 shows an apparatus for bioprocess materials storage and handling according to an embodiment.

FIG. 2 shows a protection insert according to an embodiment.

FIG. 3 shows a cassette containing a protection insert according to an embodiment.

FIG. 4 shows a protection insert according to an embodiment.

FIG. 5 shows a method according to an embodiment.

### DETAILED DESCRIPTION

This disclosure is directed to protection inserts for use in cassettes for bioprocess bags.

As used herein, “accommodate” means to at least partially surround, including having at least some coverage of opposing sides of the accommodated item. For example, a protection insert as disclosed herein is considered to be configured to “accommodate” a bioprocess bag when the bioprocess bag can be fit between opposing arms of the protection insert.

As used herein, “within” means being positioned between at least portions of another item. For example, a protection insert and a bioprocess bag can each respectively be “within” a cassette when the cassette surrounds the protection insert and/or the bioprocess bag. Further, a bioprocess bag can be considered “within” a protection insert when the bioprocess bag is positioned between the opposing arms of the protection insert.

As used herein, “transitions” between surfaces are where two or more adjacent surfaces, each having a different plane, meet or are joined to one another. Transitions between surfaces can be, for example, corners such as sharp corners or rounded, radiused, or otherwise smoothed corners.

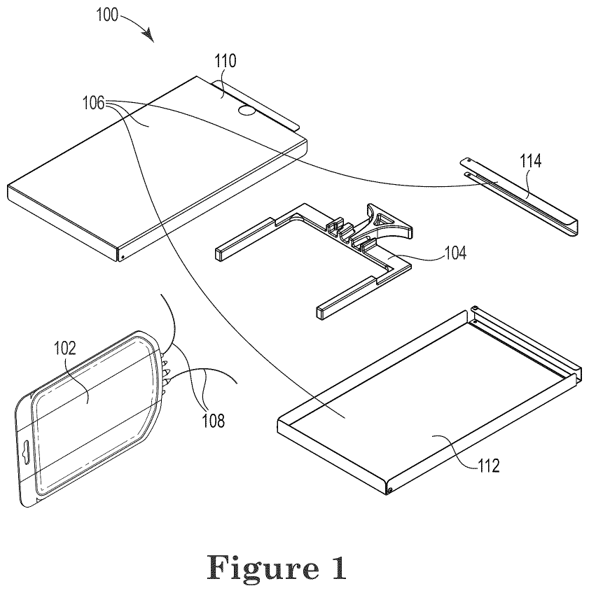

FIG. 1 shows an apparatus for bioprocess materials storage and handling according to an embodiment. Apparatus 100 includes a bioprocess bag 102, a protection insert 104, and a cassette 106.

Bioprocess bag 102 is a bag configured to contain a bioprocess material, such as drug products, proteins, blood products, vaccines, other biologic materials, buffer materials, or the like. Bioprocess bag 102 can be a bag configured for use in freezing of the bioprocess material, for example using materials suitable for cryogenic conditions. For example, bioprocess bag 102 can be configured for use at dry ice temperatures, liquid nitrogen temperatures, or the like. In an embodiment, the bioprocess bag 102 can be exposed to a temperature of −50° C. or lower. In an embodiment, the bioprocess bag 102 can be exposed to a temperature of −80° C. or lower. In an embodiment, the bioprocess bag 102 can be exposed to a temperature of −150° C. or lower. In an embodiment, the bioprocess bag 102 can be exposed to a temperature of −190° C. or lower. A non-limiting example of a bioprocess bag 102 is provided in U.S. Patent Application Pub. No. 2019/0151199, which is herein incorporated by reference in its entirety. The bioprocess bag 102 can include one or more tubes 108 extending from an end of the bioprocess bag 102. Tubes 108 can be attached to the bioprocess bag 102 through any suitable connection, such as being connected to a fitment included in bioprocess bag 102. The number of tubes 108 extending from bioprocess bag 102 can be any suitable number depending on the design of the bioprocess bag 102 and/or the contents of bioprocessing bag 102 or the processes to be performed thereon. In an embodiment, tubes 108 can be made of one or more materials that are flexible at room temperature, but that lose flexibility and become more susceptible to damage at low temperatures. Bioprocess bags 102 can have any suitable size, for example being in a range from a 2 L bag to a 50 mL bag, In an embodiment, bioprocess bags 102 can include a neck at a fitment thereof. In an embodiment, bioprocess bags 102 can be rectangular in shape.

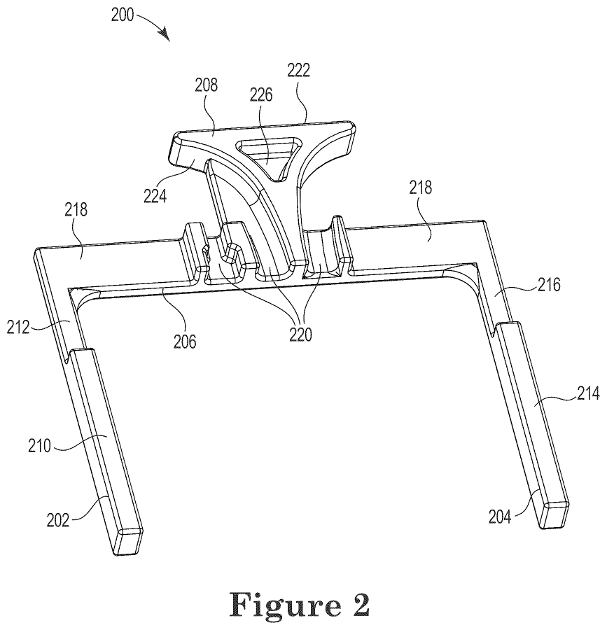

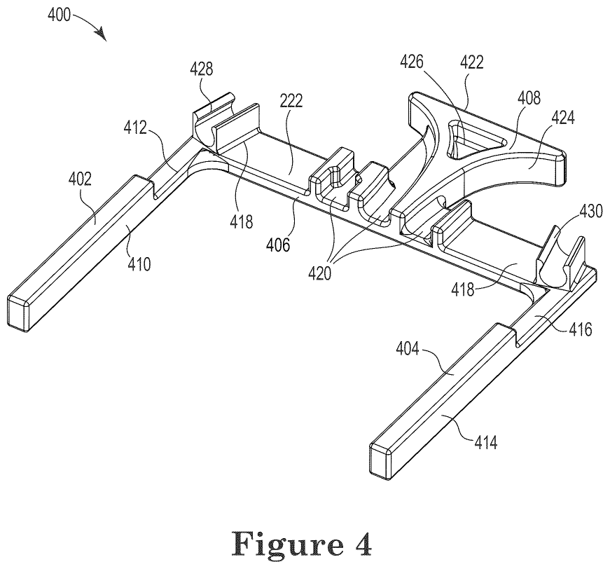

Protection insert 104 is a member configured to be placed within cassette 106 so as to secure the bioprocess bag 102 and the tubes 108 within the cassette 106. Protection inset 104 can include suitable features for engaging with an interior of the cassette 106 and accommodating the bioprocess bag 102 such that when protection insert 104 and bioprocess bag 102 are placed within cassette 106, the protection insert restricts movement of the bag 102. Protection insert 104 can be configured to direct the tubes 108 such that the tubes are curved away from an interior wall of the cassette 106. Non-limiting examples of protection inserts are shown in FIGS. 2 and 4 and discussed in further detail below.

Protection insert 104 can be reusable, for example through repeated uses in a particular cassette 102, or used in multiple different cassettes 102 over time by removing the protection insert 104 from the cassette 102 and placing the protection insert into another cassette. Protection insert 104 can be configured to be cleanable for use in a cleanroom, for example by being made of suitable materials and/or having a structure that is configured to avoid trapping water from such cleaning processes. In an embodiment, protection insert 104 can be used in processes according to Good Manufacturing Practices (GMP).

Cassette 106 is configured to accommodate the protection insert 104 and bioprocess bag 102. Cassette 106 can be configured to fully surround both the protection insert 104 and bioprocess bag 102. Cassette 106 can be configured to contain the protection insert 104 and bioprocess bag 102 during at least one processing step for the contents of the bioprocess bag 102, such as freezing and/or thawing of the contents of the bioprocess bag 102. Cassette 106 can include any suitable materials such as metals, polymers, or the like. Cassette 106 can include at least a first segment 110 and a second segment 112. Together, first segment 110 and second segment 112 can define an internal space of the cassette 106 that is capable of accommodating the bioprocess bag 102 and protection insert 104 when the first segment 110 and second segment 112 are in a closed position. The first segment 110 and second segment 112 can be at least partially separated from one another to open the cassette 106 such that the bioprocess bag 102 and/or the protection insert 104 can be removed therefrom. The cassette can further include a connector 114 connecting the first segment 110 and second segment 112. Connector 114 can facilitate the connection of first segment 110 and second segment 112, for example by providing engagement features, hinges to which the first segment 110 and/or the second segment 112 are attached, or the like.

FIG. 2 shows a protection insert according to an embodiment. Protection insert 200 includes first arm 202, second arm 204, crosspiece 206, and shielding projection 208.

Protection insert 200 is configured to be used with a cassette, such as cassette 106 described above and shown in FIG. 1. Protection insert 200 is configured to secure a position of a bag, such as bioprocess bag 102 described above and shown in FIG. 1, within the cassette. The protection insert 200 can further be configured to direct tubes extending from the bioprocess bag, such as the tubes 108 described above and shown in FIG. 1, such that the tubes are positioned away from interior walls of the cassette. In an embodiment, the protection insert can be a single unitary element. Protection insert 200 can include or be made of any suitable material, for example a polymer or a metal. Non-limiting examples of polymers that can be used in the protection insert 200 can include polycarbonate, polyethylene such as ultra-high molecular weight polyethylene (UHMW PE) or high-density polyethylene (HDPE) or the like. The width of protection insert 200 can generally correspond to the width of the internal space of the cassette, subject to manufacturing differences or variability and/or tolerances to account for the same. In an embodiment, the protection insert 200 include or be made of an elastomer.

First arm 202 is configured to extend at least partially along a side of a bioprocess bag when the bioprocess bag is positioned within protection insert 200. First arm 202 can be configured to restrict side-to-side motion of the bioprocess bag within the cassette. In an embodiment, first arm 202 can extend at least half a length of the cassette with which the protection insert 200 is configured to be used. First arm 202 can include a first bag retention region 210 having a relatively greater height than a first tube accommodation region 212 of the first arm 202. In an embodiment, the first bag retention region 210 can have a height at or near a height of the internal space of the cassette, subject to manufacturing differences or variability and/or tolerances to account for the same. The first tube accommodation region 212 can have a height such that the ends of tubes from the bioprocess bag can fit between an upper surface of the first tube accommodation region 212 and the upper surface of the internal space of the cassette. The first arm 202 can join the crosspiece 206 at the first tube accommodation region 212.

Second arm 204 is configured to extend at least partially along a side of a bioprocess bag when the bioprocess bag is positioned within protection insert 200, opposite the side along which first arm 202 is configured to extend. Second arm 204 can be configured to restrict side-to-side motion of the bioprocess bag within the cassette. In an embodiment, second arm 204 can extend at least half a length of the cassette with which the protection insert 200 is configured to be used. Second arm 204 can include a second bag retention region 214 having a relatively greater height than a second tube accommodation region 216 of the second arm 204. In an embodiment, the second bag retention region 214 can have a height at or near a height of the internal space of the cassette, subject to manufacturing differences or variability and/or tolerances to account for the same. The second tube accommodation region 216 can have a height such that the ends of tubes from the bioprocess bag can fit between an upper surface of the second tube accommodation region 216 and the upper surface of the internal space of the cassette. The second arm 204 can join the crosspiece 206 at the second tube accommodation region 216.

Crosspiece 206 extends from first arm 202 to second arm 204. Crosspiece 206 can include crosspiece tube accommodation regions 218 that are continuous with and/or have coplanar surfaces with the first and second tube accommodation regions 212, 216 of the first and second arms 202, 204. Crosspiece 206 can be configured to extend across at least a width of a bioprocess bag that protection insert 200 is configured to be used with. Crosspiece 206 can be configured to extend across an end of the bioprocess bag from which one or more tubes extend. Crosspiece 206 can include one or more tube channels 220 configured to receive at least a portion of a tube extending from the bioprocess bag, for example to retain and/or direct the tube. The tube channels 220 can each be configured to retain and/or direct different tubes extending from the bioprocess bag. The size and/or shape of the tube channels 220 can be selected based on the particular characteristics of the tubes extending from the bioprocess bag, such as the position, length, thickness, flexibility, or other such characteristics of the tubes. For example, one of the tube channels 220 can neck down to correspond with a shape of a particular tube provided on a bioprocess bag. Where there are a plurality of tube channels 220, at least some of the tube channels 220 can differ in shape. In an embodiment, at least one of the tube channels 220 can be configured to direct the tube to bend in an arc. In an embodiment, the arc can bend away from an end wall of the internal space of the cassette.

Shielding projection 208 can extend from the crosspiece 206 on a side opposite the direction in which first and second arms 202, 204 extend from said crosspiece. The shielding projection 208 can be configured to contact the end wall of the internal space of the cassette such that the crosspiece 206 is spaced apart from said end wall. The shielding projection 208 can include a contact surface 222 on the end opposite the crosspiece 206 that is configured to configured to contact the end wall of the internal space of the cassette. In an embodiment, at least one of the tube channels 220 can extend along the shielding projection 208. The shielding projection can include curved walls 224, curving outwards as the shielding projection 208 extends from crosspiece 206 to contact surface 222. At least one of the curved walls 224 can be configured to direct a tube extending through one of the tube channels 220, for example to direct the tube in the arc bending away from an end wall of the internal space of the cassette. In an embodiment, the shielding projection 208 can include a hollow portion 226, for example to reduce weight and/or material use, facilitate manufacturing, and/or provide some shock absorption when the cassette experiences a shock. The size and/or shape of the shielding projection 208 can be based on factors such as, as non-limiting examples, ensuring that tubing of the bioprocess bag can bend with bend stress being in a suitable range and/or ensuring that the bag and insert can be accommodated within the cassette.

In embodiments, the transitions from bag-facing surfaces of the protection insert 200 to adjacent surfaces having different planes can be radiused, curved, or otherwise smoothed such that no sharp corners are presented where such corners could be contacted by a bioprocess bag positioned within protection insert 200 and the cassette. The bag-facing surfaces can include the surfaces of the first and second arms 202, 204, and crosspiece 206 that define the space where the bag can be accommodated within protection insert.

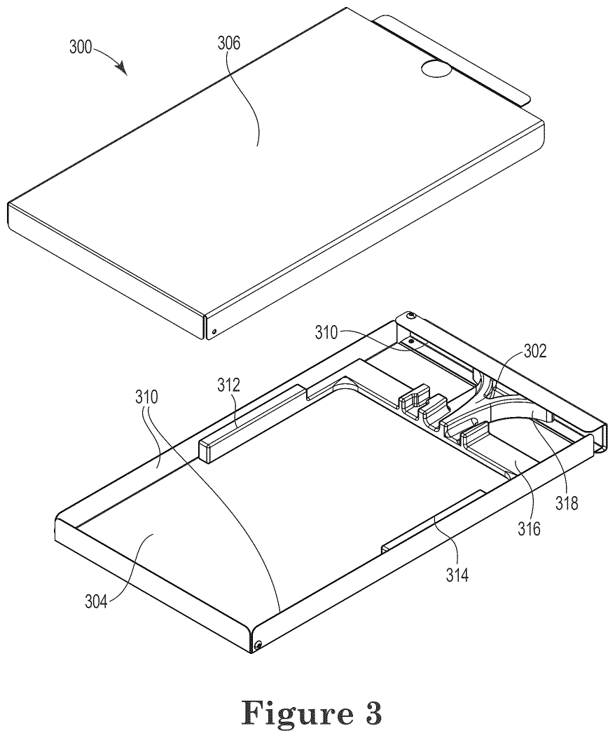

FIG. 3 shows a cassette containing a protection insert according to an embodiment. Cassette 300 includes first cassette segment 304 and second cassette segment 306. First cassette segment 304 includes an interior end wall 308 and interior side walls 310. Protection insert 302 includes first arm 312, second arm 314, crosspiece 316, and shielding projection 318 including contact surface (not shown). The contact surface of protection insert 302 can be contact surface 222 as discussed above and shown in FIG. 2. When protection insert 302 is placed into cassette 300, the first arm 312 and the second arm 314 extend along the respective interior side walls 310 of the cassette 300. The contact surface contacts interior end wall 308 such that the crosspiece 316 is spaced apart from the interior end wall 308. Accordingly, a bioprocess bag placed between the first and second arms 312, 314, and crosspiece 316 would be spaced apart from the interior end wall 308, providing space for tubes to be accommodated and protecting such tubes from being crushed if the bioprocess bag were to shift during a shock to the cassette 300. Further, the first and second arms 312, 314 would be positioned between the interior side walls 310 and the bioprocess bag.

FIG. 4 shows a protection insert according to an embodiment. Protection insert 400 includes first arm 402, second arm 404, crosspiece 406, and shielding projection 408.

Protection insert 400 is configured to be used with a cassette, such as cassette 106 described above and shown in FIG. 1. Protection insert 400 is configured to secure a position of a bag, such as bioprocess bag 102 described above and shown in FIG. 1, within the cassette. The protection insert 400 can further be configured to direct tubes extending from the bioprocess bag, such as the tubes 108 described above and shown in FIG. 1, such that the tubes are positioned away from interior walls of the cassette. In an embodiment, the protection insert can be a single unitary element. Protection insert 400 can include or be made of any suitable material, for example a polymer. Non-limiting examples of polymers that can be used in the protection insert 400 can include polycarbonate, polyethylene such as ultra-high molecular weight polyethylene (UHMW PE) or high-density polyethylene (HDPE) or the like. The width of protection insert 400 can generally correspond to the width of the internal space of the cassette, subject to manufacturing differences or variability and/or tolerances to account for the same.

First arm 402 is configured to extend at least partially along a side of a bioprocess bag when the bioprocess bag is positioned within protection insert 400. First arm 402 can be configured to restrict side-to-side motion of the bioprocess bag within the cassette. In an embodiment, first arm 402 can extend at least half a length of the cassette with which the protection insert 400 is configured to be used. First arm 402 can include a first bag retention region 410 having a relatively greater height than a first tube accommodation region 412 of the first arm 402. In an embodiment, the first bag retention region 410 can have a height at or near a height of the internal space of the cassette, subject to manufacturing differences or variability and/or tolerances to account for the same. The first tube accommodation region 412 can have a height such that the ends of tubes from the bioprocess bag can fit between an upper surface of the first tube accommodation region 412 and the upper surface of the internal space of the cassette. The first arm 402 can join the crosspiece 406 at the first tube accommodation region 412. The first tube accommodation region 412 can include a first tube retainer 428. First tube retainer 428 can be a clip, clamp, channel, or any other suitable feature for receiving a portion of a tube. The portion of the tube retained in first tube retainer 428 can be a middle or end portion of the tube. In an embodiment, the tube retained in the first tube retainer 428 is one of the tubes that extends through one of the tube channels 420. In an embodiment, the tube retained in the first tube retainer 428 is not one of the tubes that extends through one of the tube channels 420. In an embodiment, the tube retained in the first tube retainer 428 can be retained such that the tube arcs away from an end wall of the internal space of the cassette. In an embodiment, the first tube retainer 428 is configured to hold the tube in a direction angled with respect to an extending direction of the first arm 402. In an embodiment, the first tube retainer is configured to hold the tube in a direction parallel to the extending direction of the first arm 402. In an embodiment, the first tube retainer 428 can be at least partially provided on a portion of the crosspiece tube accommodation regions 418 instead of or in addition to being positioned on the first tube accommodation region 412.

Second arm 404 is configured to extend at least partially along a side of a bioprocess bag when the bioprocess bag is positioned within protection insert 400, opposite the side along which first arm 402 is configured to extend. Second arm 404 can be configured to restrict side-to-side motion of the bioprocess bag within the cassette. In an embodiment, second arm 404 can extend at least half a length of the cassette with which the protection insert 400 is configured to be used. Second arm 404 can include a second bag retention region 414 having a relatively greater height than a second tube accommodation region 416 of the second arm 404. In an embodiment, the second bag retention region 414 can have a height at or near a height of the internal space of the cassette, subject to manufacturing differences or variability and/or tolerances to account for the same. The second tube accommodation region 416 can have a height such that the ends of tubes from the bioprocess bag can fit between an upper surface of the second tube accommodation region 416 and the upper surface of the internal space of the cassette. The second arm 404 can join the crosspiece 406 at the second tube accommodation region 416. The second tube accommodation region 416 can include a second tube retainer 430. Second tube retainer 430 can be a clip, clamp, channel, or any other suitable feature for receiving a portion of a tube. The portion of the tube retained in second tube retainer 430 can be a middle or end portion of the tube. In an embodiment, the tube retained in the second tube retainer 430 is one of the tubes that extends through one of the tube channels 420. In an embodiment, the tube retained in the second tube retainer 430 is not one of the tubes that extends through one of the tube channels 420. In an embodiment, the tube retained in the second tube retainer 430 can be retained such that the tube arcs away from an end wall of the internal space of the cassette. In an embodiment, the second tube retainer 430 is configured to hold the tube in a direction angled with respect to an extending direction of the second arm 402. In an embodiment, the second tube retainer is configured to hold the tube in a direction parallel to the extending direction of the second arm 402. In an embodiment, the second tube retainer 430 can be at least partially provided on a portion of the crosspiece tube accommodation regions 418 instead of or in addition to being positioned on the second tube accommodation region 416.

Crosspiece 406 extends from first arm 402 to second arm 404. Crosspiece 406 can include crosspiece tube accommodation regions 418 that are continuous with and/or have coplanar surfaces with the first and second tube accommodation regions 412, 416 of the first and second arms 402, 404. Crosspiece 406 can be configured to extend across at least a width of a bioprocess bag that protection insert 400 is configured to be used with. Crosspiece 406 can be configured to extend across an end of the bioprocess bag from which one or more tubes extend. Crosspiece 406 can include one or more tube channels 420 configured to receive at least a portion of a tube extending from the bioprocess bag, for example to retain and/or direct the tube. The tube channels 420 can each be configured to retain and/or direct different tubes extending from the bioprocess bag. The size and/or shape of the tube channels 420 can be selected based on the particular characteristics of the tubes extending from the bioprocess bag, such as the position, length, thickness, flexibility, or other such characteristics of the tubes. For example, one of the tube channels 420 can neck down to correspond with a shape of a particular tube provided on a bioprocess bag. Where there are a plurality of tube channels 420, at least some of the tube channels 420 can differ in shape. In an embodiment, at least one of the tube channels 420 can be configured to direct the tube to bend in an arc. In an embodiment, the arc can bend away from an end wall of the internal space of the cassette.

Shielding projection 408 can extend from the crosspiece 406 on a side opposite the direction in which first and second arms 402, 404 extend from said crosspiece. The shielding projection 408 can be configured to contact the end wall of the internal space of the cassette such that the crosspiece 406 is spaced apart from said end wall. The shielding projection 408 can include a contact surface 422 on the end opposite the crosspiece 406 that is configured to configured to contact the end wall of the internal space of the cassette. In an embodiment, at least one of the tube channels 420 can extend along the shielding projection 408. The shielding projection can include curved walls 424, curving outwards as the shielding projection 408 extends from crosspiece 406 to contact surface 420. At least one of the curved walls 424 can be configured to direct a tube extending through one of the tube channels 420, for example to direct the tube in the arc bending away from an end wall of the internal space of the cassette. In an embodiment, the shielding projection 408 can include a hollow portion 426, for example to reduce weight and/or material use, facilitate manufacturing, and/or provide some shock absorption when the cassette experiences a shock.

In embodiments, the transitions from bag-facing surfaces of the protection insert 400 to adjacent surfaces having different planes can be radiused, curved, or otherwise smoothed such that no sharp corners are presented where such corners could be contacted by a bioprocess bag positioned within protection insert 400 and the cassette. The bag-facing surfaces can include the surfaces of the first and second arms 402, 404, and crosspiece 406 that define the space where the bag can be accommodated within protection insert.

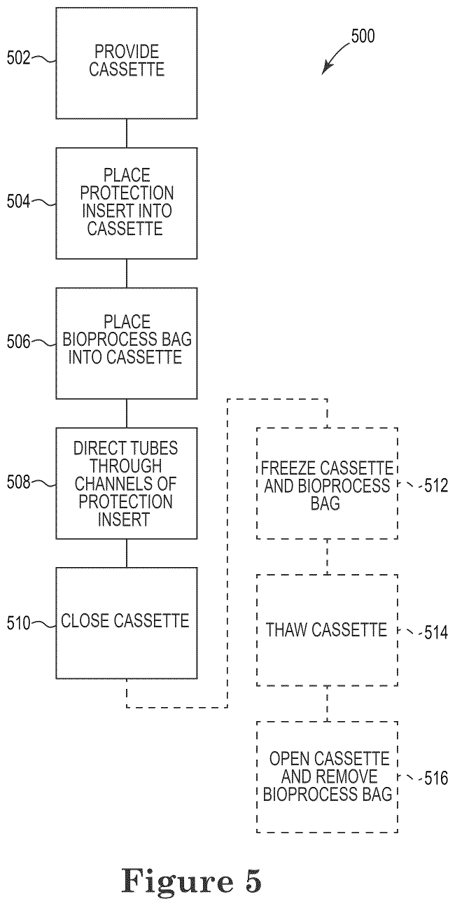

FIG. 5 shows a method according to an embodiment. Method 500 includes providing a cassette 502 and placing the protection insert into the cassette at 504. The method 500 further includes placing the bioprocess bag into the cassette at 506. Tubes connected to the bioprocess bag can be directed through tube channels on the protection insert at 508. The cassette can then be closed with the bioprocess bag contained within the cassette at 510. Optionally, the cassette can be frozen at 512 and/or thawed at 514. Optionally, the cassette can be opened and the bioprocess bag removed from the cassette at 516.

A cassette is provided at 502. The cassette can be any cassette for containing a bioprocess bag. In an embodiment, the cassette can be the cassette 104 as shown in FIG. 1 and described above.

A protection insert is placed into the cassette at 504. The protection insert can be any protection insert as described herein, such as protection insert 200 or protection insert 400 as described above and respectively shown in FIGS. 2 and 4. The protection insert can be placed into the cassette at 504 such that a contact surface at an end of a shielding projection contacts an end wall of an internal space provided in the cassette. The protection insert can be placed into the cassette at 505 such that arms of the protection insert extend along at least portions of respective side walls of the internal space provided in the cassette.

The bioprocess bag is placed into the cassette at 506. The bioprocess bag can be, for example, the bioprocess bag 102 as discussed above and shown in FIG. 1. The bioprocess bag can be filled with any suitable contents when the bioprocess bag is placed into the cassette at 506, such as drug products, proteins, blood products, vaccines, other biologic materials, buffer materials, or the like. The bioprocess bag can be placed into the cassette at 506 such that the bioprocess bag is positioned between the arms of the protection insert, with the arms of the protection insert being between the bioprocess bag and the side walls of the internal space of the cassette, and the crosspiece and/or the shielding projection of the protection insert between the end of the bag and an end wall of the internal space of the cassette.

One or more tubes connected to the bioprocess bag can be directed through one or more tube channels provided in the protection insert at 508. The tubes can be guided by the tube channels to position the tubes in a space between the bioprocess bag and an end wall of the internal space of the cassette. The tubes can each be directed into a corresponding tube channel provided in the protection insert at 508. Optionally, the tubes can further be directed into a tube retainer, such as first or second tube retainer 428, 430 as shown in FIG. 4 and described above. Directing the tubes into the tube channels of the protection insert at 508 can position the tubes such that the tubes are protected from impacts in the event of a shock, for example by having the tubes in a space between the crosspiece of the protection insert and the end wall of the internal space of the cassette. In an embodiment, once directed through the tube channels of the protection insert at 508, the tubes can extend in an arc away from the end wall of the internal space of the cassette.

The cassette is closed with the bioprocess bag and the protection insert contained within said cassette at 510. When the cassette is closed at 510, the bioprocess bag and the tubes can be secured within the cassette such that movement of the bag and/or tubes is restricted, thus reducing a risk of damage to the bag and/or the tubes in the event of a shock to the cassette while it contains the bioprocess bag, compared to a cassette containing a bioprocess bag without a protection insert.

Optionally, the method 500 can further include freezing the bioprocess bag within the cassette at 512. The freezing at 512 can be through any suitable freezing method, such as placement of the cassette into a freezer, for example a blast freezer, placement of the cassette into a cryogenic environment such as a liquid at cryogenic temperatures, or any other way of freezing the contents of the bioprocess bag within the cassette.

Optionally, the method 500 can further include thawing the bioprocess bag within the cassette at 514. In an embodiment, the thawing is performed subsequent to freezing at 512. In an embodiment, the bioprocess bag is placed into the cassette while the contents are frozen and step 514 is performed without prior freezing within the cassette at 512. The thawing can be through any suitable thawing method, such as exposing the cassette to ambient temperatures, placing the cassette into a relatively warmer fluid, directing air such as warmed air over the cassette or the like.

Optionally, the method 500 can further include removing the bioprocess bag from the cassette at 516. The cassette can be opened and the bioprocess bag removed. The removal of the bioprocess bag from the cassette at 516 can include removing the tubes from the tube channels and any tube retainers prior to removing the bioprocess bag from the cassette. The removal of the bioprocess bag from the cassette at 516 can include lifting the bioprocess bag out of the cassette once tubes have been removed from any tube retainers and/or tube channels of the protection insert.

### Aspects:

It is understood that any of aspects 1-12 can be combined with any of aspects 13-20.

Aspect 1. An apparatus, comprising:
a protection insert, the protection insert comprising:
a crosspiece;
a first arm extending from a first end of the crosspiece;
a second arm extending from a second end of the crosspiece, the crosspiece, the first arm, and the second arm configured to partially accommodate a bioprocess bag;
a shielding projection extending from the crosspiece, the shielding projection extending on a side of the crosspiece opposite extending directions of the first arm and the second arm; and
one or more tube channels provided on the crosspiece, each of the one or more tube channels configured to receive at least one of a plurality of tubes extending from the bioprocess bag.

Aspect 2. The apparatus according to aspect 1, further comprising the bioprocess bag and a cassette, the cassette configured to contain the protection insert and the bioprocess bag.

Aspect 3. The apparatus according to aspect 2, wherein the first arm and the second arm each extend at least half of a length of an internal space of the cassette.

Aspect 4. The apparatus according to any of aspects 2-3, wherein the first arm and the second arm each include a bag retention segment configured to be positioned between the bioprocess bag and at least one side wall of a cassette when the protection insert is positioned within the cassette and the bioprocess bag is positioned within the protection insert.

Aspect 5. The apparatus according to any of aspects 1-4, wherein the protection insert includes a plurality of surfaces configured to face the bioprocess bag, and transitions between said plurality of surfaces and adjacent surfaces of the protection insert are rounded.

Aspect 6. The apparatus according to any of aspects 1-5, wherein at least one of the tube channels is defined at least in part by the shielding projection.

Aspect 7. The apparatus according to any of aspects 1-6, wherein at least one of the one or more tube channels is configured to direct a respective one of the plurality of tubes extending from the bioprocess bag to follow an arc.

Aspect 8. The apparatus according to any of aspects 1-7, wherein the protection insert is formed of a polymer.

Aspect 9. The apparatus according to any of aspects 1-8, wherein the protection insert includes an elastomer.

Aspect 10. The apparatus according to any of aspects 1-9, wherein the crosspiece and at least one of the first arm and the second arm define a tube resting region.

Aspect 11. The apparatus according to aspect 10, wherein the tube resting region includes a tube retention feature.

Aspect 12. The apparatus according to any of aspects 1-11, wherein the shielding projection includes a contact surface at a distal end thereof, the contact surface configured to contact an interior wall of a cassette when the protection insert is positioned within said cassette.

Aspect 13. An apparatus, comprising:
a cassette configured to accommodate a bag; and
a protection insert comprising:
a crosspiece;
a first arm extending from a first end of the crosspiece;
a second arm extending from a second end of the crosspiece, the crosspiece, the first arm, and the second arm configured to partially accommodate the bag;
a shielding projection extending from the crosspiece, the shielding projection extending on a side of the crosspiece opposite extending directions of the first arm and the second arm; and
one or more tube channels provided on the crosspiece, each of the one or more tube channels configured to receive at least one of a plurality of tubes extending from the bag.

Aspect 14. The apparatus according to aspect 13, wherein the cassette comprises a first cassette segment and a second cassette segment, wherein the first cassette segment and the second cassette segment are joined by a hinge.

Aspect 15. The apparatus according to any of aspects 13-14, wherein the first arm and the second arm of the protection insert each extend at least half of a length of an internal space of the cassette.

Aspect 16. The apparatus according to any of aspects 13-15, wherein at least one of the one or more tube channels is configured to direct a respective one of the plurality of tubes extending from the bioprocess bag to follow an arc.

Aspect 17. The apparatus according to any of aspects 13-16, wherein the protection insert includes a plurality of surfaces configured to face the bag, and transitions between said plurality of surfaces and adjacent surfaces of the protection insert are rounded.

Aspect 18. The apparatus according to any of aspects 13-17, wherein the shielding projection includes a contact surface at a distal end thereof, the contact surface configured to contact an interior wall of a cassette when the protection insert is positioned within said cassette.

Aspect 19. The apparatus according to any of aspects 13-18, wherein the crosspiece and at least one of the first arm and the second arm define a tube resting region.

Aspect 20. The apparatus according to aspect 19, wherein the tube resting region includes a tube retention feature.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. An apparatus, comprising:
a protection insert, the protection insert comprising:
a crosspiece;
a first arm extending from a first end of the crosspiece;
a second arm extending from a second end of the crosspiece, the crosspiece, the first arm, and the second arm configured to partially accommodate a bioprocess bag;
a shielding projection extending from the crosspiece, the shielding projection extending on a side of the crosspiece opposite extending directions of the first arm and the second arm; and
one or more tube channels provided on the crosspiece, each of the one or more tube channels configured to receive at least one of a plurality of tubes extending from the bioprocess bag.

2. The apparatus of claim 1, further comprising the bioprocess bag and a cassette, the cassette configured to contain the protection insert and the bioprocess bag.

3. The apparatus of claim 2, wherein the first arm and the second arm each extend at least half of a length of an internal space of the cassette.

4. The apparatus of claim 2, wherein the first arm and the second arm each include a bag retention segment configured to be positioned between the bioprocess bag and at least one side wall of a cassette when the protection insert is positioned within the cassette and the bioprocess bag is positioned within the protection insert.

5. The apparatus of claim 1, wherein the protection insert includes a plurality of surfaces configured to face the bioprocess bag, and transitions between said plurality of surfaces and adjacent surfaces of the protection insert are rounded.

6. The apparatus of claim 1, wherein at least one of the tube channels is defined at least in part by the shielding projection.

7. The apparatus of claim 1, wherein at least one of the one or more tube channels is configured to direct a respective one of the plurality of tubes extending from the bioprocess bag to follow an arc.

8. The apparatus of claim 1, wherein the protection insert is formed of a polymer.

9. The apparatus of claim 1, wherein the protection insert includes an elastomer.

10. The apparatus of claim 1, wherein the crosspiece and at least one of the first arm and the second arm define a tube resting region.

11. The apparatus of claim 10, wherein the tube resting region includes a tube retention feature.

12. The apparatus of claim 1, wherein the shielding projection includes a contact surface at a distal end thereof, the contact surface configured to contact an interior wall of a cassette when the protection insert is positioned within said cassette.

13. An apparatus, comprising:
a cassette configured to accommodate a bag; and
a protection insert comprising:
a crosspiece;
a first arm extending from a first end of the crosspiece;
a second arm extending from a second end of the crosspiece, the crosspiece, the first arm, and the second arm configured to partially accommodate the bag;
a shielding projection extending from the crosspiece, the shielding projection extending on a side of the crosspiece opposite extending directions of the first arm and the second arm; and
one or more tube channels provided on the crosspiece, each of the one or more tube channels configured to receive at least one of a plurality of tubes extending from the bag.

14. The apparatus of claim 13, wherein the cassette comprises a first cassette segment and a second cassette segment, wherein the first cassette segment and the second cassette segment are joined by a hinge.

15. The apparatus of claim 13, wherein the first arm and the second arm of the protection insert each extend at least half of a length of an internal space of the cassette.

16. The apparatus of claim 13, wherein at least one of the one or more tube channels is configured to direct a respective one of the plurality of tubes extending from the bioprocess bag to follow an arc.

17. The apparatus of claim 13, wherein the protection insert includes a plurality of surfaces configured to face the bag, and transitions between said plurality of surfaces and adjacent surfaces of the protection insert are rounded.

18. The apparatus of claim 13, wherein the shielding projection includes a contact surface at a distal end thereof, the contact surface configured to contact an interior wall of a cassette when the protection insert is positioned within said cassette.

19. The apparatus of claim 13, wherein the crosspiece and at least one of the first arm and the second arm define a tube resting region.

20. The apparatus of claim 19, wherein the tube resting region includes a tube retention feature.
